(19) United States
(12) Patent Application Publication
(10) Pub. No.: US 2012/0052538 A1
(43) Pub. Date: Mar. 1, 2012

(54) TRIGLYCERIDES WITH HIGH CONTENT OF UNSATURATED FATTY ACIDS

(75) Inventors: Per Munk Nielsen, Hilleroed (DK); Jesper Brask, Bagsvaerd (DK); Steffen Ernst, Broenshoej (DK)

(73) Assignee: NOVOZYMES A/S, Bagsvaerd (DK)

(21) Appl. No.: 13/262,002

(22) PCT Filed: Mar. 29, 2010
(86) PCT No.: PCT/EP10/54137

§ 371 Date: Oct. 25, 2011

(30) Foreign Application Priority Data
Apr. 6, 2009 (EP) 09157372.5

(51) Int. Cl. C12P 7/64

(52) U.S. Cl. 435/134

(57) ABSTRACT

The invention relates to a process for producing a triglyceride product comprising the steps of: (a) subjecting a triglyceride feedstock comprising at least 25 mole %, based on the total amount of acyl groups in the triglycerides, unsaturated C18 fatty acid residues to an alcoholy- sis reaction with an alcohol having 1 to 6 carbon atoms (C1-C6) to obtain a reaction mixture, 5 wherein the conversion is at least 65%; (b) separating from the reaction mixture produced in step (a) fractions comprising (i) unsaturated C18 fatty acid alkyl esters and (ii) glycerides; and (c) using the fractions (i) and (ii) or subfractions thereof in a condensation reaction catalyzed by a lipase to produce a triglyceride product comprising at least 80 mole % unsaturated fatty acids.

### FIELD OF THE INVENTION

The invention relates to the field of lipids. It relates to the manufacturing of glycerides by employing lipases. More particularly, the invention relates to a process for producing unsaturated triglycerides from recycled fractions.

### BACKGROUND OF THE INVENTION

Diets with high levels of saturated lipids are known to raise blood cholesterol and to increase the risk of cardiac vascular diseases. It is therefore desirable to decrease the amount of saturated lipids and to increase the amount of unsaturated lipids in consumer products.

Several reports describing the manufacture of triglycerides by enzymatic catalysis to obtain unsaturated lipids have been published. In particular, within the field of fish oil: WO 95/24459 (Norsk Hydro A/S) describes a process applied to fish oils where a fraction enriched in glycerides with polyunsaturated fatty acids such as EPA, DHA and AA is separated from a fraction with saturated fatty acids and monounsaturated glycerides. They do not recombine fractions to form triglycerides but proceed with alcoholysis until essentially all fatty acids are esterified. U.S. Pat. No. 6,905,850 B2 (Nippon Suisan Kaisha, Ltd) also relates to fish oils. It describes a process for producing triglycerides having polyunsaturated fatty acids in the 2-position and medium-chain saturated fatty acid residues having the carbon number of 8, 10 or 12 at the 1- and 3-positions. A method to produce triacylglycerol from marine n-3 PUFA and glycerol was suggested by Kosugi & Azuma 1994 (Synthesis of triacylglycerol from polyunsaturated fatty acid by immobilized lipase. JAOCS Vol. 71 no. 12) where pure free fatty acids or ethyl esters thereof were reacted with glycerol in a lipase catalysed condensation reaction. They achieved high content of PUFA triglycerides.

Most manufacturing processes are based on modification of lipids which initially contain a certain amount of a fatty acid residue which is desirable in the end product. These lipids are enzymatically modified to generate a mixture from where desired fraction(s) containing the intermediate or end product may be isolated. Thus, end product yield and production cost are highly influenced by the lipid feedstock. Furthermore, depending on the lipid feedstock various amount and/or fractions may be left unexploited.

Hernandez-Martin and Otero (2009), J. Agric. Food Chem., 57, pp. 701-708, discloses selective elimination of saturated fatty acid residues from triacylglycerol constituting soybean oil followed by reesterification of the monoacylglycerol and diacylglycerol thus obtained with conjugated linoleic acid (CLA) obtained from somewhere else. This results in lipids essentially free of saturated fatty acids and rich in essential unsaturated fatty acids. However, the reaction setup is dependent on CLA supplied from another source, which adds significantly to the cost of the lipids produced.

Accordingly, there is still a desire to develop efficient and less expensive processes to modify triglycerides by reducing the content of saturated lipids and thereby obtain consumer products with increased amounts of unsaturated lipids.

### SUMMARY OF THE INVENTION

The present invention relates to a process for producing a triglyceride product comprising the steps of: (a) subjecting a triglyceride feedstock comprising at least 25 mole %, based on the total amount of acyl groups in the triglycerides, unsaturated C18 fatty acid residues to an alcoholysis reaction with an alcohol having 1 to 6 carbon atoms (C₁-C₆) to obtain a reaction mixture, wherein the conversion is at least 65%; (b) separating from the reaction mixture produced in step (a) fractions comprising (i) unsaturated C18 fatty acid alkyl esters and (ii) glycerides; and (c) using the fractions (i) and (ii) or subfractions thereof in a condensation reaction catalyzed by a lipase to produce a triglyceride product comprising at least 80 mole % unsaturated fatty acids.

One advantage of such process is a very efficient use of the triglyceride feedstock. The triglyceride product obtained from step (c) can be used in products for human consumption and has a higher value in such products than the triglyceride feedstock. And saturated fatty acid alkyl esters obtained as a by-product can be used, e.g., as fuel. Further, the process can proceed without further supply of unsaturated fatty acids from another source.

### DETAILED DESCRIPTION OF THE INVENTION

The terms to be defined below are shown in capitals and have been listed alphabetically:

ALCOHOLYSIS is the reaction between an alcohol and a glyceride such as an oil or fat. If the alcohol concerned is ethanol the alcoholysis can also be referred to as ethanolysis, if methanol is employed the alcoholysis can also be referred to as ‘methanolysis’, etc.

CONDENSATION is defined as the reaction between an ester and glycerol where alcohol and glyceride is formed.

CONVERSION is defined as the molar fraction of fatty acids in the glycerides structure of the raw material that have been reacted by the enzyme catalyzed reaction. This can be measured by mol. For transesterification of glycerides with ethanol: Conversion=FAEE/FAIG, where FAEE=mol Fatty Acid Ethyl Ester after reaction and FAIG=mol Fatty Acids in glycerides before reaction. For hydrolysis of glycerides: Conversion=(FFA_{end}−FFAₛₜₐᵣₜ)/FAIG, where FFA_{end}=mol Free Fatty Acids after reaction, FFAₛₜₐᵣₜ=mol Free Fatty Acids in raw material before the reaction, and FAIG=mol Fatty Acids in glycerides before reaction.

ESTERIFICATION is the reaction between a fatty acid and an alcohol leading to an ester and water.

HYDROLYSIS is the reaction between an ester and water and is the reversible reaction of esterification.

FATTY ACID DISTILLATE is the condensate resulting from a vapour scrubbing process during the vacuum stripping of triglyceride oils which latter process is used for the physical removal of free fatty acids and for the deodorisation of triglyceride oils. In addition to FFA or FFA esters, the fatty acid distillate contains unsaponifiables such as but not limited to tocopherols and sterols.

FATTY FEED is a general name for raw materials containing fatty acid moieties. These can be glycerides such as monoacylglyceride, also referred to as monoglyceride, diglycerides, triglycerides and phosphatides but free fatty acids and even soaps can form part of the fatty feed.

FFA is the standard abbreviation of Free Fatty Acids.

OLEIN of oil or a fat product is the low-melting fraction obtained by solid/liquid separation of the product at a temperature where part of the content is solidified.

TRANSESTERIFICATION is the reaction between a glyceride having R1 and a fatty acid having R2 whereby the R-groups is exchanged leading to a glyceride having R2 and a fatty acid having R1.

### Process of the Invention

The object of the present invention is to provide a high efficient process for producing a high purity grade triglyceride product which is enriched in unsaturated fatty acids relative to the triglyceride feedstock. The aim is furthermore to generate other high purity grade products such as e.g. saturated fatty acid alkyl esters for e.g. biodiesel and/or unsaturated fatty acid alkyl esters, in particular monounsaturated which may be recycled in the process of the invention. It is asserted that glycerides including mono-, di- and tri-glycerides, fatty acids, fatty acid alkyl esters, glycerol, and alcohol products obtained by said process have a high purity chemical grade or high purity food grade. Furthermore, the object of the present invention is to provide a process for producing a triglyceride product comprising at least 80 mole % unsaturated fatty acids. The process is based on the recycling of separated fractions in said process to obtain the triglyceride product.

In some embodiments the invention relates to a process for producing a triglyceride product comprising the steps of: (a) subjecting a triglyceride feedstock comprising at least 25 mole %, based on the total amount of acyl groups in the triglycerides, unsaturated C18 fatty acid residues to an alcoholysis reaction with an alcohol having 1 to 6 carbon atoms (C₁-C₆) to obtain a reaction mixture, wherein the conversion is at least 65%; (b) separating from the reaction mixture produced in step (a) fractions comprising (i) unsaturated C18 fatty acid alkyl esters and (ii) glycerides; and (c) using the fractions (i) and (ii) or subfractions thereof in a condensation reaction catalyzed by a lipase to produce a triglyceride product comprising at least 80 mole % unsaturated fatty acids.

An alcoholysis reaction (step a) is conducted by bringing the starting material i.e. triglyceride feedstock and alcohol together with one mole triglycerides to at least 3*0.65 mole alcohol. The reaction may be conducted at temperatures of 25-60° C., 30-55° C., 35-50° C., or 35-45° C. The reaction may be catalyzed by a chemical catalyst or by an enzymatic catalyst such as a lipase. Lipases suitable for use in the process of the invention are described in more details in the section “Lipases”. Catalysts which may be soluble or immobilized are added in concentrations high enough to enable conversion within 24 to 48 hours. The reaction may take place in a batch reaction, packed bed, or fluidized bed system.

The conversion of the alcoholysis reaction is at least 65%; at least 70%; at least 75%; at least 80%; at least 82%; at least 84%; at least 86%, at least 88%, at least 90%; at least 92%; at least 94%; at least 96% or at least 98%.

Triglyceride feedstock, alcohol and enzyme are not easily miscible and this obstacle has in the past been overcome by the addition of a solvent. However the process according to the present invention is devoid of any solvent.

### Lipid Feedstock

Any lipid i.e. oils and fats of vegetable or animal origin comprising triglycerides may be used as feedstock in the process of the invention and as basis for producing the triglyceride product. The triglyceride feedstock may comprise polar lipids such as phospholipids; and non-polar lipids such as triglycerides, diglycerides, and monoglycerides; or any combination thereof.

In some embodiments the invention relates to a process, wherein the triglyceride feedstock is a vegetable oil.

In some embodiments the invention relates to a process, wherein the triglyceride feedstock may be selected from the group containing: Butterfat; cocoa butter; corn; lard; olive; palm; palm kernel; peanut; rapeseed; rice bran; coconut; cottonseed; grape seed; sesame; soybean; sunflower; tallow; whale; including fractions derived thereof; or any combination thereof.

Depending on the triglyceride feedstock the content of unsaturated C18 fatty acid residues may vary, and thus in some embodiments the invention relates to a process, wherein the starting triglycerides comprises at least 30 mole %; at least 35 mole %; at least 40 mole %; at least 45 mole %; at least 50 mole %; at least 55 mole %; at least 60 mole %; at least 65 mole %; at least 70 mole %; at least 75 mole % unsaturated C18 fatty acid residues. In other embodiments the invention relates to a process, wherein the starting triglycerides comprises at most 80 mole %; at most 75 mole %; at most 70 mole %; at most 65 mole %; at most 60 mole %; at most 55 mole %; at most 50 mole %; at most 45 mole %; at most 40 mole %; at most 35 mole % unsaturated C18 fatty acid residues.

### Alcohol

Alcohols used in the process of the invention are alcohols having 1 to 6 carbon atoms (C₁-C₆) like e.g. methanol; ethanol; propanol; butanol; pentanol; or hexanol. They are preferably monohydric alcohols. They may be primary alcohols such as methanol (MeOH), ethanol (EtOH), 1-propanol (PrOH), 1-butanol (n-BuOH), and 1-pentanol, 1-hexanol (OH); secondary alcohols such as 2-propanol (iPrOH); tertiary alcohols; or any combination thereof. Thus the structure of the alcohol may be linear/straight or branched. Preferably, the alcohol is methanol (MeOH) and/or ethanol (EtOH).

In some embodiments the invention relates to a process, wherein the alcohol having 1 to 6 carbon atoms (C₁-C₆) is selected from the group consisting of: methanol; ethanol; propanol; butanol; isobutanol; pentanol; pentanediol; isopentanol; hexanol; and any combination thereof.

It has surprisingly been found that a certain amount of water content is permissible. Both absolute (99%) as well as 96% EtOH (azeotroph) may be used in the process of the invention as is apparent from examples 1 and 2. Thus, the alcohol which has not been utilized (unreacted) in the alcoholysis reaction as well as the alcohol generated in the condensation reaction of the invention may be recycled. The alcohol may be separated from the reaction mixture and/or separated from the condensation reaction mixture for reuse in the alcoholysis reaction of the process of the invention.

In some embodiments the invention relates to a process, wherein alcohol having 1 to 6 carbon atoms (C₁-C₆) may be obtained from unreacted alcohol isolated from the alcoholysis reaction mixture, and/or from generated alcohol isolated from the condensation reaction mixture, and recycled to step (a).

### Methods of Separation

The separation (step b) is conducted to obtain the fraction of C18:x alkyl esters (where 0<x<3) required to meet the specifications of the triglyceride product and/or the end-product. The moles of C18:x must be larger than the number of positions on the glycerides where the esters can be bound in the condensation reaction.

The alcoholysis reaction mixture may contain without being limiting unsaturated as well as saturated C18 fatty acid alkyl esters; glycerides; unreacted alcohol; glycerol; and unsaturated as well as saturated C16 fatty acid alkyl esters.

In some embodiments the separation is done to obtain a fraction comprising unsaturated C18 fatty acid alkyl esters required to meet the specifications of the triglyceride product i.e. the end-product.

Separation of fractions may be conducted using methods known in the art such as crystallization, deodorization, distillation, centrifugation, evaporation, membrane filtration, membrane separation, molecular distillation, short path distillation, phase separation by gravity, molecular sieve, stripping, thermal fractionation, urea precipitation etc.

CRYSTALLISATION is used here to describe solid/liquid separation processes based on differences in melting points, i.e. carried out at a temperature where some compounds of a mixture are solid and some are not. Crystallization is also referred to as thermal fractionation and both terms are used interchangeably.

DEODORISATION is essentially a steam distillation under vacuum.

DISTILLATION is the process of heating a liquid to its boiling point and condensing and collecting the vapor in liquid form. Short path distillation is a special construction of the distillation unit that secures a low pressure drop in the equipment.

EVAPORATION is a process step converting at least one component to the vapor form. Evaporation comprises specific forms such as distillation and deodorization.

MEMBRANE SEPARATION designates processes, by which liquid/liquid separation of different molecular species is secured by semi-permeable membranes.

MOLECULAR DISTILLATION is distillation in high vacuum, intended to make possible use of low temperatures to protect heat-labile compounds.

STRIPPING, also referred to as vacuum stripping when carried out at sub-atmospheric pressure, is a process that causes the most volatile constituents of a mixture to vaporize when a gas is blown through the mixture.

THERMAL FRACTIONATION is another term for crystallization.

In some embodiments the invention relates to a process, wherein the separation of fractions is conducted with a method selected from the group containing: distillation, crystallization, centrifugation, urea precipitation, membrane filtration; molecular sieve; or any combination thereof.

Separation may be distillation after which further separation methods may be conducted such as fractionation (crystallisation) or urea sedimentation of the light fraction from the distillation to further increase the content of C18:x. Thus, in some embodiments the invention relates to a process, wherein separation of the fraction comprising C18 fatty acid alkyl esters is conducted by the method of distillation and/or crystallization.

After distillation glycerol and glycerides are comprised in the same fraction and the two components may be divided into separate fractions by centrifugation. Alternatively, this combined fraction comprising both glycerol and glycerides may be used directly in step (c) without further separation.”

In some embodiments the invention relates to a process, wherein separation of the fraction comprising glycerides is conducted by the method of distillation and centrifugation, and in some embodiments the invention relates to a process, wherein separation of the fraction comprising glycerol is conducted by the method of distillation and centrifugation.

In some embodiments the invention relates to a process, wherein separation of the fraction comprising alcohol is performed by distillation optionally followed by molecular sieve absorption of water. This fraction may be re-cycled and used as a starting material.

In some embodiments the invention relates to a process, wherein separation of the fraction comprising C16 fatty acid alkyl esters is conducted by the method of distillation and/or crystallization. This fraction may if necessary be further separated to obtain a saturated C16:0 fraction which may be used as a separate by-product such as for fuel (biodiesel).

In some embodiments the invention relates to a process, wherein at least one further fraction is separated from the reaction mixture selected from the group containing fractions comprising: (iii) alcohol; (iv) glycerol; and (v) C16 fatty acid alkyl esters.

It is to be understood that the alcohol thus obtained may be re-used in step (a), and the glycerol thus obtained may be used in step (c).

The C18 fatty acid residue fraction may depending of the triglyceride feedstock contain saturated C18:0 fatty acid residues and unsaturated C18:x fatty acid residues such as monounsaturated 018:1 fatty acid residues, and poly-unsaturated C18:p fatty acid residues like diunsaturated 018:2 fatty acid residues; tri-unsaturated C18:3 fatty acid residues etc.

In some embodiments the invention relates to a process, wherein the fraction comprising unsaturated C18 fatty acid alkyl esters is further enriched in unsaturated C18 fatty acid alkyl esters by removal of saturated C18 fatty acid alkyl esters.

In some embodiments the invention relates to process, wherein the fraction comprising unsaturated C18 fatty acid alkyl esters is further enriched in specific unsaturated C18 fatty acid alkyl esters where the specific unsaturated C18 fatty acid alkyl esters are selected from the group containing: C18:1 (mono-unsaturated) fatty acid alkyl esters; C18:2 (di-unsaturated) fatty acid alkyl esters; C18:3 (tri-unsaturated) fatty acid alkyl esters; or any combination thereof.

In some embodiments the invention relates to a process, wherein the fraction of unsaturated C18 fatty acid acyl esters is further separated to generate at least one fraction selected from the group containing: C18:1 (mono-unsaturated) fatty acid alkyl esters; 018:2 (di-unsaturated) fatty acid alkyl esters; or C18:3 (tri-unsaturated) fatty acid alkyl esters.

In some embodiments the invention relates to a process wherein the fatty acid residues have a chain length below 24 carbon atoms (C24); below 22 carbon atoms (C22); or below 20 carbon atoms (C20).

In some embodiments the invention relates to a process wherein there are below 10; below 9; below 8; below 7; below 6; below 5; below 4; below 3; or below 2 double bonds present in a fatty acid residue.

### Condensation Reaction

The condensation reaction (step c) is conducted by bringing the fractions or subfractions of (i) C18 fatty acid alkyl esters and (ii) glycerides together in the presence of a lipase whereby the triglyceride product is formed. The triglyceride product may be used directly or it may be further refined to obtain an end-product. The amount of unsaturated C18 fatty acid alkyl esters must be larger than the number of positions in the glycerides, including di- and monoglycerides that are available for binding of these esters during the condensation reaction. This is important to ensure formation of triglycerides. The desired stoichometry may be obtained by calculating and subsequently adjusting the amount of unsaturated fatty acid alkyl esters and glycerides that are used in the condensation reaction.

The alcohol may continuously be evaporated during condensation and optionally be recycled and used in the alcoholysis reaction.

Unconverted fatty acid alkyl esters may optionally be added to the next batch of condensation (i.e., recycled) or separated off for other purposes or discarded.

As an alternative, the alcoholysis reaction mixture may be depleted of the fractions (iv) glycerol and/or (v) C16 fatty acid alkyl esters prior to addition of a lipase for conduction a condensation reaction. It is to be understood that it is also possible to maintain at least part of the glycerol in the mixture which is subjected to the condensation reaction.

At distillation the alcohol will be separated off first (e.g. EtOH has a boiling point at 79° C. at 750 mm Hg). The C16 fatty acid alkyl esters (e.g. Ethyl palmitate 303° C. at 750 mm Hg, 192-193° C. at 10 mm Hg) has a lower boiling point than C18 fatty acid alkyl ester (Ethyl oleate 205-208° C. at 750 mm Hg, 216-218° C. at 15 mm Hg; Ethyl stearate 213-215° C. at 15 mm Hg; Ethyl linoleate 224° C. at 15 mm Hg) and will during distillation be separated of first. By separating off the C16 fraction as well as sufficient amount of glycerol to balance the stochiometry, the remaining components comprising the C18 fraction and the glyceride fraction may be submitted to a condensation reaction to generate the triglyceride product.

### Lipases

Lipases are enzymatic catalysts that may be used in the alcoholysis reaction as well as in the condensation reaction.

Lipases as used herein means an enzyme that has lipase activity, generally classified as EC 3.1.1.x, and catalyze reactions such as hydrolysis, interesterification, transesterefication, esterification, alcoholysis, acidolysis and aminolysis. Lipases particularly relevant for the present invention are those that catalyze the esterification of fatty acids or transesterification of fatty acid esters in the presence of alcohol to yield fatty acid alkyl ester.

Phospholipases, a subgroup of lipases, as used herein means an enzyme that has phospholipase activity said enzyme may catalyze reactions that lead to the formation of fatty acid alkyl ester and is for the purpose of the present invention also defined as a lipase. Where the triglycerid feedstock comprises phospholipids, phospholipases is particularly relevant to include in the alcoholysis reaction.

Acyltransferase as used herein means an enzyme which as well as having lipase activity (generally classified as EC 3.1.1.x) also has acyltransferase activity, generally classified as EC 2.3.1.X, whereby the enzyme is capable of transferring an acyl group from an acyl donor to one or more acyl acceptor substrates selected from: any compound comprising a hydroxyl group (—OH) i.e. alcohols such as sterol, stanol, glycerol etc; carbohydrate; protein; protein subunit. Lipid acyltransferase catalyzes reactions such as transesterification and alcoholysis, and thus said enzyme may catalyze reactions that lead to the formation of fatty acid alkyl ester and is for the purpose of the present invention also defined as a lipase. Reactions may in the presence of water in certain embodiments of the invention be catalyzed by acyltransferase.

Lipases, suitable for use in a process and/or contained in a composition of the invention, may be obtained from microorganisms, such as filamentous fungus, yeast, or bacteria. In some embodiments the lipases may be formulated as immobilized products as will be described further below.

For the purpose of the present invention the term “obtained from”, as used herein in connection with a specific microbial source, means that the enzyme and consequently the DNA sequence encoding said enzyme is produced by the specific source. The enzyme is then obtained from said specific source by standard known methods enabling the skilled person to obtain a sample comprising the enzyme and capable of being used in a process of the invention. Said standard methods may be direct purification from said specific source or cloning of a DNA sequence encoding the enzyme followed by recombinant expression either in the same source (homologous recombinant expression) or in a different source (heterologous recombinant expression).

Most lipases used as catalysts in organic synthesis are of microbial and fungal origin, and these are readily available by fermentation and basic purification. Lipases extracted from various sources have successfully been used in processes for generating biodiesel. Candida Antarctica B lipase immobilized on acrylic resin (Novozym 435) has been the most commonly used enzyme in experiments for the production of biodiesel. However, depending on experimental variables such as substrate, alcohol, water, temperature, pH, re-use etc. different lipases may be utilized.

In certain embodiments the present invention relates to a process, wherein the lipase is selected from the group containing: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype or thologs and homologs thereof; and variants thereof that have an amino acid sequence that is at least 60%; at least 70%, at least 75%; at least 80%, at least 85%; at least 90%, at least 92%; at least 94%; at least 95%, at least 96%; at least 97%; at least 98% or at least 99% identical to any of those wildtype enzymes.

In certain embodiments the present invention relates to a process, wherein the lipase is selected from the group deposited in NCBI's Genebank database as accession numbers: YP_{—}890535 (GID: 118468600 as also described in WO05/056782; M. smegmatis); NP_{—}436338.1 (GID: 16263545; Sinorhizobium meliloti); ZP_{—}01549788.1 (GID: 118592396; Stappia aggregate); NP_{—}066659.1 (GID: 10954724; Agrobacterium rhizogenes); YP_{—}368715.1 (GID: 78065946; Burkholderia sp); YP_{—}674187.1 (GID: 110633979; Mesorhizobium sp.); NP_{—}532123.1 (GID: 17935333; Agrobacterium tumefaciens); Agrobacterium rhizogenes (Q9 KWA6); A. rhizogenes (Q9 KWB1); A. tumefaciens (Q8UFG4); A. tumefaciens (Q8UAC0); A. tumefaciens (Q9Z109); A. tumefaciens (ACA); Prosthecobacter dejongeii (RVM04532); Rhizobium. loti (Q98MY5); R. meliloti (Q92XZ1); R. meliloti (Q9EV56), R. rhizogenes (NF006), R. rhizogenes (NF00602875), R. solanacerarum (Q8XQI0); Sinorhizobium meliloti (RSM02162); Sinorhizobium meliloti (RSM05666); Mesorhizobium loti (RMLO00301); A. rhizogenes (Q9 KWA6); A. rhizogenes (Q9 KWB1); Agrobacterium tumefaciens (AAD02335); Mesorhizobium loti (Q98MY5); Mesorhizobium loti (ZPOO 197751); Ralstonia solanacearum (Q8XQI0); Ralstonia eutropha (ZPOO 166901); Moraxella bovis (AAK53448); Burkholderia cepacia (ZP00216984); Chromobacterium violaceum (Q7NRP5); Pirellula sp. (NP_{—}865746); Vibrio vulnificus (AA007232); Salmonella typhimurium (AAC38796); Sinorhizobium meliloti (SMal993); Sinorhizobium meliloti (Q92XZ1); Sinorhizobium meliloti (Q9EV56); and wildtype orthologs and homologs thereof; and variants thereof that have an amino acid sequence that is at least 60%; at least 70%, at least 75%; at least 80%, at least 85%; at least 90%, at least 92%; at least 94%; at least 95%, at least 96%; at least 97%; at least 98% or at least 99% identical to any of those wildtype enzymes.

The identity may be calculated based on either amino acid sequences or nucleotide sequences.

The relatedness between two amino acid sequences or between two nucleotide sequences is described by the parameter “identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

### Immobilized Lipases

The use of immobilized lipases in oils and fats processing are experiencing significant growth due to new technology developments that have enabled cost effective methods. A fundamental advantage of immobilized lipases is that they can be recovered and reused from a batch process by simple filtration. Further, packing of immobilized lipases in columns allows for easy implementation of a continuous process. Immobilized enzymes generally also have a positive effect on operational stability of the catalyst (compared to free enzymes), it makes handling easier (compared to free enzyme powder), and it allows operation under low-water conditions (compared to liquid formulated enzymes).

Various ways of immobilizing lipases are well known in the art. A review of lipase immobilization is found in “Immobilized lipase reactors for modification of fats and oils—a review” Malcata, F X., et al. (1990) Journal of American Oil Chemist's Society Vol. 67 p. 890-910, where examples of representative lipase immobilizing carriers are illustrated, including inorganic carriers such as diatomaceous earth, silica, porous glass, etc.; various synthetic resins and synthetic resin ion exchangers; and natural polysaccharide carriers such as cellulose and cross-linked dextrin.

In some embodiments the invention relates to process, wherein the lipase is immobilized.

In some embodiments the invention relates to a process, wherein the lipase is covalently or non-covalently immobilized on a carrier; or alternatively by entrapment in natural or synthetic matrices, such as sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In some embodiments the invention relates to a process, wherein the carrier is a hydrophilic carrier selected from the group containing: porous in-organic particles composed of alumina, silica and silicates such as porous glas, zeolites, diatomaceous earth, bentonite, vermiculite, hydrotalcite; and porous organic particles composed of carbohydrate polymers such as agarose or cellulose.

In some embodiments the invention relates to a process, wherein the carrier is a hydrophobic carrier selected from the group containing: synthetic polymers such as polyacrylates, polymethacrylates, nylon, polyethylene, polypropylene or polystyrene; and activated carbon. Many synthetic hydrophobic polymer carriers are (crosslinked) copolymers containing several different monomer components.

Lipases in solid form, such as immobilized lipases, may be used in some embodiments of the invention and examples of commercially available immobilized lipases include the ones sold under the trade names LIPOZYME™ TL IM, LIPOZYME™ RM IM, and Novozym™ 435 (Novozymes A/S).

In case the reaction is carried out with liquid formulations of a lipase (in contrast to an immobilized lipase) the enzyme can be recovered for multiple uses by either separation off the aqueous/glycerol phase containing the enzyme or by using a membrane reactor. In a membrane reactor the end-product is separated from the lipase by using a membrane filtration system.

### Triglyceride Product

In some embodiments the invention relates to a process, wherein at least 88%; at least 90%, at least 92%; at least 94%; at least 95%; at least 96%; at least 97%; at least 98%; or at least 99% triglyceride product is obtained. Such percentage is to be understood as the percentage of total fatty acids which are incorporated into a triglyceride in the final product (mole %).

Diets with high levels of saturated lipids are known to raise blood cholesterol and to increase the risk of cardiac vascular diseases. It is therefore desirable to decrease the amount of saturated lipids and to increase the amount of unsaturated lipids in consumer products.

The amount of unsaturated fatty acids in the triglyceride product is at least 80 mole %; preferably at least 82.5 mole %; at least 85 mole %; at least 87.5 mole %; at least 90 mole %; at least 92.5 mole %; at least 95 mole % or at least 97.2 mole % based on the total amount of acyl groups in the triglycerides.

In some embodiments the invention relates to a process, wherein the amount of saturated fatty acids in the triglyceride product is below 20 mole %; below 17.5 mole %; below 15 mole %; below 12.5 mole %; below 10 mole %, below 7.5 mole %; or below 5 mole % based on the total amount of acyl groups in the triglycerides.

A reduction of polyunsaturated fatty acid residues is known to increase the stability of some consumer products such as e.g. frying medium and it is therefore desirable to obtain products high in monounsaturated fatty acids and low in saturated and/or polyunsaturated fatty acids. For the sake of end-product stability the number of double bonds in the fatty acids should be as low as possible and preferably be limited to one or two.

In some embodiments the invention relates to a process, wherein the amount of C18:1 (mono-unsaturated) fatty acid residues is higher than the amount of C18:p (poly-unsaturated) fatty acid residues in the triglyceride product.

In some embodiments the invention relates to a process, wherein the amount of C18:p (polyunsaturated) fatty acid residues in the triglyceride product is below 50 mole %; below 40 mole %; below 30 mole %; below 20 mole %; below 15 mole %; below 10 mole %; below 7.5 mole %; below 5 mole %; below 2.5 mole %; or below 1 mole % based on the total amount of unsaturated acyl groups in the triglycerides.

In some embodiments the invention relates to a process, wherein the (poly-unsaturated) fatty acid residues in the triglyceride product contain below 6; below 5; below 4; below 3; or below 2 double bonds.

Food grade quality products are products that can be approved for human consumption by the food grade quality authorities. In some embodiments the invention relates to a process, wherein the triglyceride product is of food grade quality.

In some embodiments the invention relates to a triglyceride product obtainable by the process.

In some embodiments the invention relates to use of the triglyceride product for producing consumer products and/or fried food products preferably edible oil, consumer oil, margarine, shortenings, frying oil, battered fried products, baked products like bread, cake, cookies, biscuits or snack foods such as e.g. chips and French fries.

### Other Products

Fatty acid alkyl esters of saturated fatty acids which is a result from a methanolysis reaction is called fatty acid methyl esters (FAME) or biodiesel. BIODIESEL is defined as esters of long chain fatty acids derived from renewable feed stocks and C₁-C₃ monohydric alcohols. Examples of such renewable feed stocks are vegetable oils and animal fats. In the context of the present invention long chain fatty acids may be defined as fatty acid chains with a length of between 10 and 22 carbon atoms.

In some embodiments the invention relates to a process, wherein a fraction of saturated fatty acid alkyl esters are separated from the reaction mixture.

In some embodiments the invention relates to use of the fraction of saturated fatty acid alkyl ester as fuel or fuel additive.

Glycerol may as described be isolated for the purpose of generating a high purity grade glycerol by-product. Unconverted C18 may be isolated from the condensation reaction mixture for the purpose of being reused in a new round of condensation.

The present invention is further described by the following examples that should not be construed as limiting the scope of the invention.

### Materials and Methods

Chemicals used as buffers and substrates were commercial products of at least reagent grade. All chemicals, unless otherwise indicated, were obtained from Sigma-Aldrich or a similar commercial source and used without further purification.

### Example 1

### Production of Fatty Acid Alkyl Esters

A transesterification experiment was carried out as a 2⁴-factorial screening trial (two levels and four factors). The Design of Experiment function in the statistical software SAS JMP® version 8.0 from SAS Institute Inc. SAS Campus Drive, Building S, Cary, N.C., 27513, USA (see www.imp.com) was used to setup the experiment according to the parameters listed in Table 1. Data in table 3 are analyzed using the same software omitting 3-factor and higher interactions.

TABLE 1
Experimental setup

| | Low level | High level |
|---|---|---|
| Alcohol total 1.5eqv | methanol | ethanol |
| Candida B lipase | 4% of oil Novozym 435 | 6% of oil CalB on |
| | (hydrophobic carrier | silica carrier |
| Lipozyme TL IM | 0% | 3% of oil |
| Water | 0% | 5% |

The lipase activity of 4% of Novozym 435 corresponds to the lipase activity of 6% of CaIB on silica carrier

TABLE 2
Fatty acid composition of Palm olein
(Aarhus Karlshamns, Aarhus, Denmark)

| Fatty acid | w/w % |
|---|---|
| C6:0 Hexanoic acid | <0.1 |
| C8:0 Octanoic acid | <0.1 |
| C10:0 Decanoic acid | <0.1 |
| C12:0 Lauric acid | 0.5 |
| C14:0 Myristic acid | 1.1 |
| C15:0 Pentadecanoic acid | 0.1 |
| C16:0 Palmitic acid | 39.6 |
| C16:1 cis/sum | 0.2 |
| C16:1 trans/sum | <0.1 |
| C17:0 Heptadecanoic acid | 0.1 |
| C18:0 Stearic acid | 4.3 |
| C18:1 cis/sum | 41.8 |
| C18:1 trans/sum | 0.1 |
| C18:2 cis/sum | 11.0 |
| C18:2 trans/sum | 0.2 |
| C18:3 cis/sum | 0.3 |
| C18:3 trans/sum | 0.1 |
| C20:0 Arachidic acid | 0.4 |
| C20:1 cis/sum | 0.2 |
| C20:2 cis/sum | <0.1 |
| C22:0 Behenic acid | 0.1 |
| C22:1 cis/sum | <0.1 |
| C24:0 Lignoceric acid | <0.1 |
| C24:1 cis/sum | <0.1 |

The composition comprises 53.5% w/w unsaturated C18 fatty acids.

25 g palm olein was added to a 250 ml bottle together with alcohol, enzyme, and water according to the amounts indicated in Table 1 and the reactants according to Table 3. Alcohol was added as 0.5 molar equivalents at time 0 hour and 1 molar equivalent at 7 hour. The samples were incubated with shaking at 35° C. Total reaction time was 30 hours and samples for analysis were taken at the time points 7, 24, and 30 hours.

The amounts of esters produced were measured by nuclear magnetic resonance (NMR) and the amounts of free fatty acids (FFA) were measured by titration.

TABLE 3
Conversion (%) of fatty acid alkyl esters and free fatty acids

| | | | Lipo- | | | | | | | |
|---|---|---|---|---|---|---|---|---|---|---|
| | | | zyme | | Es- | Es- | Es- | | | |
| | Alco- | | TL | | ter | ter | ter | FFA | FFA | FFA |
| No. | hol | CaIB | IM | H₂O | 7 h | 24 h | 30 h | 7 h | 24 h | 30 h |
| 1 | EtOH | H | − | − | 30 | 42 | 39 | −0.2 | 0.2 | 0.4 |
| 2 | EtOH | H | − | + | 8 | 14 | 13 | 0.0 | 0.7 | 1.1 |
| 3 | EtOH | H | + | − | 57 | 92 | 92 | 1.5 | 0.5 | 0.6 |
| 4 | EtOH | H | + | + | 64 | 88 | 89 | 22.0 | 4.0 | 4.6 |
| 5 | EtOH | S | − | − | 33 | 40 | 38 | 0.1 | 0.2 | 0.3 |
| 6 | EtOH | S | − | + | 6 | 11 | 10 | 0.4 | 0.5 | 0.6 |
| 7 | EtOH | S | + | − | 58 | 74 | 70 | 1.0 | 0.4 | 0.4 |
| 8 | EtOH | S | + | + | 10 | 30 | 30 | 1.8 | 1.3 | 2.0 |
| 9 | MeOH | H | − | − | 37 | 40 | 41 | 0.0 | 0.2 | 0.1 |
| 10 | MeOH | H | − | + | 16 | 19 | 24 | 0.5 | 0.5 | 2.1 |
| 11 | MeOH | H | + | − | 46 | 62 | 60 | 1.1 | 0.5 | 0.6 |
| 12 | MeOH | H | + | + | 46 | 102 | 102 | 26.8 | 2.4 | 2.6 |
| 13 | MeOH | S | − | − | 36 | 37 | 38 | 0.2 | 0.2 | 0.5 |
| 14 | MeOH | S | − | + | 9 | 12 | 12 | 0.1 | 0.5 | 0.6 |
| 15 | MeOH | S | + | − | 43 | 48 | 48 | 1.1 | 0.8 | 3.0 |
| 16 | MeOH | S | + | + | 34 | 57 | 56 | 5.5 | 1.6 | 3.1 |

Silica carrier (S); Hydrofobic carrier (H).

Statistical analysis of the data obtained was performed using the SAS JMP program Based on this analysis the following conclusions were made:

No significant effect of the type of alcohol used could be observed.

The type of carrier for CaIB had no influence of the degree of conversion.

The water content tested did not influence the amount of alkyl ester formed after 24 hours reaction time. This observation supports the possibility of reusing alcohol as a 95% alcohol which eliminates the need for drying the azeotroph after distillation.

Combining the CaIB enzyme with Lipozyme TL IM improved the conversion rate significantly.

The statistical model predicts that the amount of fatty acid alkyl ester formed after a 24 hour reaction time with 3% Lipozyme TL IM, 4% Novozym 435 is 85%.

### Example 2

### Fatty Acid Alkyl Esters May be Generated in the Presence of Water

The effect of ethanol dosage on the formation of ethyl esters was tested using 96% ethanol. Temperature of reaction was 35° C., 25 g palm olein and 2 g Lipozyme TL IM. Ethanol was added in different dosages, i.e. 0.75 equivalents, 1 equivalent and 5 equivalents corresponding to the amount of fatty acids in the oil. The ethanol was added all from the start. Amount of esters formed after 24 hours were measured by NMR and used to calculate the % conversion:

0.75 eq.=67%
1 eq.=73%
5 eq.=74%

The results showed that the increase of ethanol dosage from 0.75 eq to 1 eq resulted in higher conversion, whereas 5 eq. gave the same result as 1 eq.

### Example 3

### Production of Triglycerides from Fatty Acid Alkyl Esters

Condensation experiments were performed as batch reactions either under vacuum conditions (50° C. heated water batch with magnetic stirring about 300 rpm, vacuum about 30 mbar and a connected freezing trap) or assisted by N₂-bubbling (column reactor with 50° C. heating jacket and a N₂-up flow) to facilitate continuous removal of formed ethanol. 100 g substrate: ethyl oleate (FAE)+glycerol and/or partial glycerides (MG/DG) were reacted in stoichiometric amounts (gly:FA 1:3 molar ratio, where gly is the total molar amount of glycerol and glyceride backbone from MG/DG, and where FA is the total molar amount of fatty acids from FAE and MG/DG). FAE+enzyme (5 wt % immobilized: Lipozyme435 or 1 wt % liquid: Lipozyme CALB L) were weighted into the flasks and equilibrated to reaction temperature. The reaction was started by drip wise addition of glycerol and/or MG/DG through a drip regulating funnel with pressure equalisation (vacuum driven systems) or a piston pump (N₂-bubbling system) at a flow rate about 0.3 g/min. The reactions were continued up to 93 hours with samples withdrawal as the reaction progressed (0.5-1 mL). Samples were stored at 5° C. until analysis for FAE content by GC method: EN-14103 and glyceride distribution by a modified form of the TLC-FID procedure described in Journal of Molecular Catalysis B: Enzymatic 28 (2004) 19-24. Results (Table 4) shows up to 99% TG after 48-76 hours of reaction. Both operation modes (N₂-bubbling as well as magnetic stirring under vacuum) were found suited for enzymatic re-assembly of TG. Immobilized enzyme (Lipozyme435) was shown to be more efficient than the corresponding liquid enzyme (CALB L). Partial glycerides reacted more readily than glycerol with the fatty acid alkyl esters.

TABLE 4
FAE conversion & glyceride formation during condensation

| Reactants | | | Con- | Reaction time (hours) | | | | | | | | | | |
|---|---|---|---|---|---|---|---|---|---|---|---|---|---|---|
| (gly:FA = | | Reac- | tent | | | | | | | 24- | | 48- | | |
| 1:3) | Enzyme | tor | (wt %) | 0 | 2 | 4-5 | 10 | 17 | 20 | 25 | 28 | 52 | 76 | 93 |
| Glycerol + | NZ435 | Vac- | FAE | 100 | 79.2 | 44.8 | 23.5 | | 13.2 | | 7.4 | 2.6 | 1.8 | |
| FAE | | uum | TG | 0.0 | 3.3 | 11.4 | 40.2 | | 78.9 | | 81.7 | 91.0 | 94.9 | |
| | | batch | DG | 0.0 | 13.0 | 38.0 | 35.1 | | 7.1 | | 10.0 | 5.4 | 3.1 | |
| | | | MG | 0.0 | 4.5 | 5.9 | 1.2 | | 0.8 | | 0.9 | 1.0 | 0.2 | |
| | | | SUM | 100 | 100 | 100 | 100 | | 100 | | 100 | 100 | 100 | |
| Glycerol + | CALB L | Vac- | FAE | 100 | 94.5 | 93.2 | | | 65.9 | | 57.8 | 42.4 | | |
| FAE | | uum | TG* | 0.0 | 5.5 | 6.8 | | | 34.1 | | 42.2 | 57.6 | | |
| | | batch | | | | | | | | | | | | |
| Glycerol + | NZ435 | N2- | FAE | 100 | 87.4 | | | 25.0 | | 9.6 | | | | 0.0 |
| FAE | | bub- | TG | 0.0 | 2.0 | | | 47.1 | | 56.8 | | | | 74.2 |
| | | bling | DG | 0 | 7.6 | | | 27.9 | | 28.2 | | | | 23.0 |
| | | col- | MG | 0 | 3.0 | | | 0 | | 5.4 | | | | 2.8 |
| | | umn | SUM | 100 | 100 | | | 100 | | 100 | | | | 100 |
| MG/DG + | NZ435 | Vac- | FAE | 100 | | 57.5 | | | | 10.1 | 6.9 | 2.3 | | |
| Glycerol | | uum | TG* | 0 | | 42.5 | | | | 89.9 | 93.1 | 97.7 | | |
| (85:15 | | batch | | | | | | | | | | | | |
| wt %) + | | | | | | | | | | | | | | |
| FAE | | | | | | | | | | | | | | |
| MG/DG + | NZ435 | Vac- | FAE | 100 | | 26.4 | | | | 5.1 | 3.7 | 1.3 | | |
| FAE | | uum | TG* | 0 | | 73.6 | | | | 94.9 | 96.3 | 98.7 | | |
| | | batch | | | | | | | | | | | | |

*Based on FAE conversion

### Example 4

A palm olein raw material with the fatty acid composition of 45% saturated fatty acids and 55% unsaturated fatty acids is used as the raw material for production of triglyceride product with less than 15% saturated fatty acids.

In a batch reactor 100 kg of the olein is heated to 35° C. and added lipase (Lipozyme TL immobilized on hydrophobic carrier for instance Lewatit) in a dosage of 10% w/w of the olein. Addition of ethanol is started. In total 16.3 kg ethanol (96%) is added during 3 hours. When more than 90% of the fatty acids in the olein has been converted to ethyl esters the reaction is terminated. It is expected to be within less than 4 hours.

The reaction mixture is now containing the immobilized enzyme, fatty acid ethyl esters, glycerol, ethanol, water, and small amounts of mono, di-, and tri-glycerides. The liquid fraction is separated from the enzymes by a sieve.

The sieved fraction is centrifuged to eliminate glycerol/water as the heavy fraction.

The light fraction from the centrifugation is distilled to obtain four fractions:

a) an ester fraction enriched in C18-esters
b) an ester fraction enriched in C16-esters
c) a residue consisting mainly of MG, DG and TG
d) Ethanol which is re-circulated to the next batch.

The C18-ester fraction is characterized by having preferably at least 85% fatty acids as unsaturated fatty acids, or more preferably at least 90% fatty acids as un-saturated fatty acids, or more preferably at least 95% fatty acids as un-saturated fatty acids.

The C18-ester fraction is mixed with the MG, DG, and TG fraction from distillation and with dry glycerol where the amount of FFA is in a stoichiometrically surplus. The mixture is added a reactor with 10% w/w to the reaction mixture C. antarctica lipase B (e.g. Novozym 435) at 50° C. The glycerol is added gradually over a period of 4 hours. The reactor is designed in a way to eliminate the ethanol formed from the condensation reaction. This can be done either by vacuum or by bubbling nitrogen through the reaction mixture. The reaction continues until a content of triglycerides of at least 94%. Reaction time can be as long as 48 hours depending on the efficiency of ethanol removal. The ethanol from the condensation reaction can be collected to be used in the next batch.

After the reaction the end-product is deodorized to eliminate FFA, Fatty acid esters and residual ethanol.

1-15. (canceled)

16. A process for producing a triglyceride product comprising the steps of:
a) subjecting a triglyceride feedstock comprising at least 25 mole %, based on the total amount of acyl groups in the triglycerides, unsaturated C18 fatty acid residues to an alcoholysis reaction with an alcohol having 1 to 6 carbon atoms (C1-C6) to obtain a reaction mixture, wherein the conversion is at least 65%;
b) separating from the reaction mixture produced in step (a) fractions comprising (i) unsaturated C18 fatty acid alkyl esters and (ii) glycerides; and
c) using the fractions (i) and (ii) or subfractions thereof in a condensation reaction catalyzed by a lipase to produce a triglyceride product comprising at least 80 mole % unsaturated fatty acids.

17. The process of claim 16, wherein step (a) is catalyzed by a lipase.

18. The process of claim 16, wherein the triglyceride feedstock is a vegetable oil.

19. The process of claim 16, wherein the alcohol having 1 to 6 carbon atoms (C1-C6) is selected from the group consisting of: methanol; ethanol; propanol; butanol; isobutanol; pentanol; pentanediol; isopentanol; hexanol; and any combination thereof.

20. The process of claim 16, wherein at least 50% of the alcohol having 1 to 6 carbon atoms (C1-C6) is obtained from unreacted alcohol isolated from the alcoholysis reaction mixture, and/or from generated alcohol isolated from the condensation reaction mixture, and recycled to step (a).

21. The process of claim 16, wherein the fraction comprising unsaturated C18 fatty acid alkyl esters is further enriched in unsaturated C18 fatty acid alkyl esters by removal of saturated C18 fatty acid alkyl esters.

22. The process of claim 16, wherein the fraction comprising unsaturated C18 fatty acid alkyl esters is further enriched in specific unsaturated C18 fatty acid alkyl esters selected from the group consisting of: C18:1 (mono-unsaturated) fatty acid alkyl esters; C18:2 (di-unsaturated) fatty acid alkyl esters; C18:3 (tri-unsaturated) fatty acid alkyl esters; and any combination thereof.

23. The process of claim 16, wherein at least one further fraction is separated from the reaction mixture, where the at least one further fraction is selected from the group consisting of: (iii) alcohol; (iv) glycerol; and (v) C16 fatty acid alkyl esters.

24. The process of claim 16, wherein the separation of fractions is conducted with a method selected from the group consisting of: distillation, crystallization, centrifugation, urea precipitation, membrane filtration; molecular sieve; and any combination thereof.

25. The process of claim 16, wherein the lipase used in step (c) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 60% identical to any of those wildtype enzymes.

26. The process of claim 16, wherein the lipase in step (c) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 90% identical to any of those wildtype enzymes.

27. The process of claim 16, wherein the lipase in step (c) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 95% identical to any of those wildtype enzymes.

28. The process of claim 16, wherein the lipase in step (c) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 98% identical to any of those wildtype enzymes.

29. The process of claim 16, wherein the lipase used in step (a) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 60% identical to any of those wildtype enzymes.

30. The process of claim 16, wherein the lipase used in step (a) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 90% identical to any of those wildtype enzymes.

31. The process of claim 16, wherein the lipase used in step (a) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 95% identical to any of those wildtype enzymes.

32. The process of claim 16, wherein the lipase used in step (a) is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Candida rugosa lipase; Corynebacterium acnes lipase; Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof that have an amino acid sequence that is at least 98% identical to any of those wildtype enzymes.

33. The process of claim 17, wherein the lipase is immobilized.

34. The process of claim 16, wherein the amount of C18:1 (monounsaturated) fatty acid residues is higher than the amount of C18:p (poly-unsaturated) fatty acid residues in the triglyceride product.

35. The process of claim 16, wherein a fraction of saturated fatty acid alkyl esters are separated from the reaction mixture.
